(19) United States
(12) Patent Application Publication
(10) Pub. No.: US 2026/0325732 A1
(43) Pub. Date: Oct. 22, 2026

(54) Filter Device and Method for Stroke Prevention in Patients Undergoing Complex Thoracic Aortic Interventions

(71) Applicant: Baptist Health South Florida, Inc., Coral Gables, FL (US)

(72) Inventors: Barry T. Katzen, Miami Beach, FL (US); Juan Carlos Parodi, Miami Beach, FL (US)

(21) Appl. No.: 19/643,446

(22) Filed: Apr. 9, 2026

Related U.S. Application Data
(60) Provisional application No. 63/785,809, filed on Apr. 9, 2025.

(51) Int. Cl. A61M 1/36

(52) U.S. Cl. CPC A61M 1/3659; A61M 1/3633; A61M 2202/0021; A61M 2202/0028; A61M 2202/0042; A61M 2202/005; A61M 2202/0413; A61M 2202/06; A61M 2205/3331; A61M 2205/7545; A61M 2210/12

(57) ABSTRACT

A filter device for filtering particles from blood flow generated during aortic interventions in order to reduce a risk of neurological deficit includes a venous sheath with a connector and a side port and an arterial sheath with a connector and a side port. The filter device also includes a filter assembly connected between the side port of the venous sheath and the side port of the arterial sheath. The filter assembly includes a housing or enclosure with a fluid inlet, a fluid outlet, a filter disposed within the housing or enclosure between the inlet and the outlet and three-way stopcocks disposed at the inlet and the outlet of the enclosure for controlling blood from through the filter assembly between the arterial sheath and the venous sheath.

### CROSS REFERENCE TO RELATED APPLICATION

This application claims the benefit of United States Provisional Patent Application No. 63/785,809, filed on Apr. 9, 2025, the disclosure of which is hereby incorporated by reference in its entirety.

### BACKGROUND OF THE INVENTION

### Field of the Invention

The present disclosure is generally directed to blood filter devices and, in particular, to filter devices including stopcock valves for controlling blood flow in order to capture particles generated during a surgical procedure and to surgical methods for deployment and use of such filter devices.

### Description of Related Art

Aortic interventions include procedures such as percutaneous valve replacement and insertion of endografts in the aorta. Instrumentation used during such procedures can produce particles which can migrate through vasculature to the brain causing neurological deficits, such as asymptomatic brain ischemia and stroke. Vascular filter devices are known which can be deployed to capture such particles. However, there is a need in the art for improved filter devices and surgical methods in order to increase efficiency in capturing generated particles thereby reducing risks to patients associated with such aortic interventions.

### SUMMARY OF THE INVENTION

According to an aspect of the disclosure, a filter device for filtering particles from blood flow generated during aortic interventions in order to reduce a risk of neurological deficit includes a venous sheath with a connector and a side port and an arterial sheath with a connector and a side port. The filter device also includes a filter assembly connected between the side port of the venous sheath and the side port of the arterial sheath. The filter assembly includes a housing or enclosure with a fluid inlet, a fluid outlet, a filter disposed within the housing or enclosure between the inlet and the outlet and three-way stopcocks disposed at the inlet and the outlet of the enclosure for controlling blood from through the filter assembly between the arterial sheath and the venous sheath.

According to another aspect of the disclosure, a surgical method for capturing particles generated during an aortic intervention with the filter device, as previously described, includes a step of, prior to commencing the aortic intervention, inserting the arterial sheath of the filter device into a femoral artery in a retrograde fashion. The method also includes steps of: inserting the venous sheath into a vein in a cephalad direction; connecting the filter assembly between the side port of the venous sheath and the side port of the arterial sheath, such that the filter is disposed between the venous sheath and the arterial sheath; and after connecting the filter assembly between the side port of the venous sheath and the side port of the arterial sheath, opening the stopcocks disposed at the inlet of the housing or enclosure and at the outlet of the housing or enclosure thereby allowing blood to flow from the artery to the vein. Blood flow in this direction allows for capturing particles generated during the aortic intervention before the particles migrate to the brain, thereby reducing occurrence of or preventing stroke or another neurological deficit.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limit of the invention.

FIG. 1 is a schematic drawing of a filter device according to an aspect of the present disclosure; and

FIG. 2 is a flow chart showing a surgical method for deployment and use of a filter device according to an aspect of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. As used herein, the term “proximal” refers to a portion of a tool or device that is grasped by a user and/or is farthest away from a patient. For example, a “proximal” end of a catheter can refer to an end of the catheter that is held by a surgeon and/or connected to a fluid delivery device. By contrast, the term “distal” refers to an end of a tool or device that is opposite the proximal end. The distal end of the device can be an end that is farthest away from the portion grasped by the user. For example, for an indwelling catheter, the distal end can be the end which is inserted into the patient through an insertion site. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising,” “comprise” or “comprised,” and variations thereof, are meant to be open ended. As used herein, the term “patient” or “subject” refers to members of the animal kingdom including but not limited to human beings.

The present disclosure is directed to a filter device 10 for filtering particles from blood flow generated during aortic interventions, such as percutaneous valve replacement and/or any insertion of endograft(s) in the aorta, in order to reduce risks of neurological deficits, such as brain ischemia and stroke, caused by migration of particles to the brain generated by instrumentation. When implanted between an artery and vein, the filter device 10 is configured to control or restrict flow from the artery to the vein. Because of the flow gradient (artery to vein), flow in the aorta can be reversed allowing all or most particles generated from the aortic intervention to be captured by the filter device 10. Indwelling portions of the filter device 10 can be introduced through a large (e.g., 20 Fr or more) introducer sheath through the femoral artery in a retrograde fashion. The filter device 10 can include an optional occlusion balloon configured to be deployed in an artery for anchoring the filter device 10 in place at a desired location.

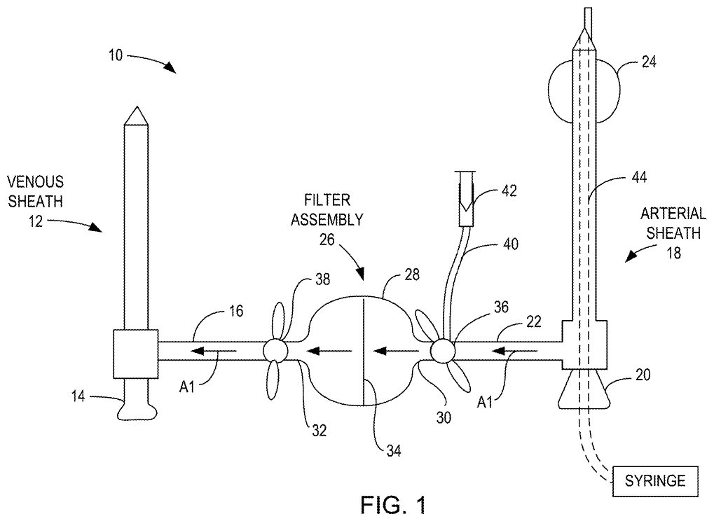

As shown in FIG. 1, the filter device 10 comprises a venous sheath 12 (e.g., a sheath of about 9 Fr or 10 Fr) that comprises a wide connector 14 and a large side port 16, which can be about 7 mm to 9 mm in diameter. The venous sheath 12 can be introduced in the cephalad direction. The filter device 10 further comprises an arterial sheath 18 (e.g., a sheath of about 20 Fr or 25 Fr) that comprises a wide connector 20 and a large (e.g., about 7 mm to 9 mm) side port 22. As discussed above, the arterial sheath 18 can optionally further comprise an occlusion balloon 24 on the arterial sheath 18 for positioning the arterial sheath 18 at a desired location. The arterial sheath 18 can be introduced in the femoral artery in a retrograde fashion.

The filter device 10 further comprises a filter assembly 26 connected between the venous sheath side port 16 and the arterial sheath side port 22. Connections between the side ports 16, 22 and the filter assembly 26 should be large (at least about 6 mm in diameter) to decrease flow resistance through lumen(s) of the side ports 16, 22 and filter assembly 26. The filter assembly 26 comprises a housing or enclosure 28 comprising a fluid inlet 30, a fluid outlet 32, and a filter 34 disposed within the housing or enclosure 28 between the inlet 30 and outlet 32. The filter assembly 26 also comprises an inflow or proximal three-way stopcock 36 at the inlet 30 of the housing or enclosure 28 and an outflow or distal three-way stopcock 38 at the outlet 32 for controlling blood flow from the artery to the vein. In some examples, a third lumen of the proximal three-way stopcock 36 can be connected to a tube 40 connected to a syringe 42 (e.g., a 50 cc syringe) for providing aspiration during critical steps of a surgical procedure, thereby increasing flow through the filter assembly 26 so that more particles can be captured by the filter 34.

In some examples, the filter device 10 can further comprise an inner sheath 44 (e.g., a sheath smaller than about 20 Fr, such as about 12 Fr) extending through a main lumen of the arterial sheath 18, which can also be used for aspiration during critical steps of a surgical procedure. As shown in FIG. 1, the inner sheath 44 can extend beyond a distal end of the arterial sheath by a short distance (e.g., about 1 cm). The inner sheath 44 can be connected at a proximal end to a syringe (e.g., a 50 cc syringe) for aspiration of fluid through the inner sheath 44 during the surgical procedure.

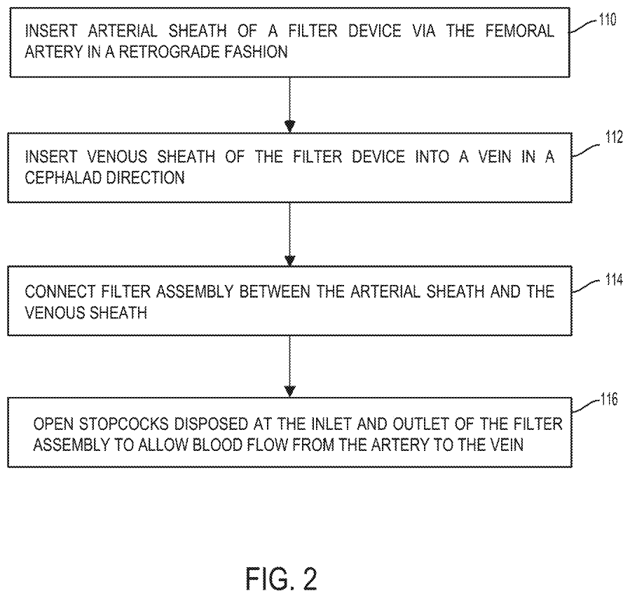

FIG. 2 is a flow chart showing steps of a method for insertion and use of the filter device 10 during an aortic intervention (e.g., percutaneous valve replacement and/or insertion of endograft(s) in the aorta). As shown at step 110, the method comprises, prior to beginning the aortic intervention procedure, inserting the arterial sheath 18 of the filter device 10 in the femoral artery in a retrograde fashion. As discussed above, components of the filter device 10 including the arterial sheath 18 and/or filter assembly 26 can be introduced through a large introducer sheath (e.g., a sheath of 20 Fr or larger). In some examples, insertion of the arterial sheath 18 can include deploying the optional occlusion balloon 24 for positioning the arterial sheath 18 in the artery. At step 112, the method further comprises inserting the venous sheath 12 into a vein in a cephalad direction. At step 114, the method comprises connecting components of the filter device 10 together. In particular, as previously described, the filter assembly 26 can be connected between the venous sheath side port 16 and the arterial sheath side port 22, such that the filter 34 is disposed within the housing or enclosure 28 and between the venous sheath 12 and the arterial sheath 18. When the components of the filter assembly 26 are connected together and inserted in the artery and vein, at step 116, the method comprises opening the stopcocks 36, 38 disposed at the inlet 30 and outlet 32 of the housing or enclosure 28 of the filter assembly 26, thereby allowing blood to flow from the artery to the vein as shown by arrow A1 in FIG. 1. As previously described, due to this gradient (e.g., artery to vein), flow in the aorta is reversed meaning that all or most of the particles generated from instrumentation of an aortic surgical intervention are captured by the filter 34 disposed in the housing or enclosure 28. Capturing these particles prevents the particles generated during the instrumentation from migrating to the brain thereby reducing occurrence of or entirely preventing stroke or any neurological deficit and, in particular, reducing a risk of asymptomatic brain ischemia.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment.

The invention claimed is:

1. A filter device for filtering particles from blood flow generated during aortic interventions in order to reduce a risk of neurological deficit, the filter device comprising:
a venous sheath comprising a connector and a side port;
an arterial sheath comprising a connector and a side port; and
a filter assembly connected between the side port of the venous sheath and the side port of the arterial sheath comprising:
a housing or enclosure comprising a fluid inlet, a fluid outlet, a filter disposed within the housing or enclosure between the inlet and the outlet; and
three-way stopcocks disposed at the inlet and the outlet of the enclosure for controlling blood from through the filter assembly between the arterial sheath and the venous sheath.

2. A surgical method for capturing particles generated during an aortic intervention with the filter device of claim 1, the method comprising:
prior to commencing the aortic intervention, inserting the arterial sheath of the filter device into a femoral artery in a retrograde fashion;
inserting the venous sheath into a vein in a cephalad direction;
connecting the filter assembly between the side port of the venous sheath and the side port of the arterial sheath, such that the filter is disposed between the venous sheath and the arterial sheath; and
after connecting the filter assembly between the side port of the venous sheath and the side port of the arterial sheath, opening the stopcocks disposed at the inlet of the housing or enclosure and at the outlet of the housing or enclosure thereby allowing blood to flow from the artery to the vein and capturing particles generated during the aortic intervention before the particles migrate to the brain, thereby reducing occurrence of or preventing stroke or another neurological deficit.
